(19) United States
(12) Patent Application Publication
(10) Pub. No.: US 2007/0021356 A1
(43) Pub. Date: Jan. 25, 2007

(54) Preemptive prophlyaxis of migraine

(76) Inventor: Roger K. Cady, Ozark, MO (US)

Correspondence Address: HUSCH & EPPENBERGER, LLC, 190 CARONDELET PLAZA, SUITE 600, ST. LOUIS, MO, 63105-3441, US

(21) Appl. No.: 10/569,148

(22) PCT Filed: Mar. 14, 2003
(86) PCT No.: PCT/US03/07993

§ 371 Date: Feb. 21, 2006

Related U.S. Application Data
(60) Provisional application No. 60/365,691, filed on Mar. 18, 2002.

(51) Int. Cl. A61K 31/7008; A61K 31/58; A61K 31/55; A61K 31/4166; A61K 31/425

(52) U.S. Cl. 514/23; 514/217; 514/373; 514/389; 514/561; 514/176

(57) ABSTRACT

A method of preventing the headache phase of migraine in a human comprises administration of an anti-convulsant medication to said human exhibiting prodrome symptoms of migraine. Suitably, the method comprises administration of a migraine headache phase-preventing effective amount of the anti-convulsant. There is also disclosed a pharmaceutical composition for the prevention of the headache phase of a migraine containing an anti-convulsant as an active ingredient. There is also disclosed a method of determining prodromal symptoms of migraine using the following cognitive tests: Simple Reaction Time (103); Running Memory Continuous Performance Task (104); Matching to Sample (105); Mathematical Processing Task (106); and interpreting the results as a percent of baseline indicator of need for prophylaxis.

### CROSS-REFERENCE

This application claims the benefit and priority of U.S. provisional application Ser. No. 60/365,691, filed 18 Mar. 2002.

### TECHNICAL FIELD

The present invention relates generally to the medical field and, more particularly, to a method for predicting the onset of a migraine headache and to a preemptive prophylaxis of the migraine headache.

The preemptive prophylaxis is directed to prevent or reduce the headache phase and/or disability of migraine in humans by the administration of drugs during the prodrome phase of migraine.

### DESCRIPTION OF THE RELATED ART

A headache may be one of several different varieties, each of which has its own unique pain characteristics which differ dramatically. The types of headache include tension, sinus, cluster, rebound and migraine. Migraine is a particularly painful headache that recurs from time to time. The pain is quite severe and often the person with migraine must stay in bed. Dietary, emotional and environmental factors may trigger an attack. On average, migraine sufferers experience an attack per month. Attacks last from four to seventy-two hours. Of interest is that the incidence of migraine appears to be on the rise. Because of the severity and incidence of migraine, prescription medicines have been invented to provide relief.

Migraine sufferers sometimes get a warning signal before the onset of the headache phase of a migraine. The warning signals apparent to the migraineur are classified as aura. The period of aura is preceded by a period classified as prodromal or premonitory period. The periods of aura, prodrome and premonitory are pre-headache. The International Headache Society (IHS) defines aura as neurological symptoms that usually develop over 5-20 minutes and last less than 60 minutes. Headache may occur immediately after aura or after an aura free interval of less than 60 minutes. Aura symptoms commonly include, but are not limited to, visual disturbances and numbness or tingling sensations. Less than 20% of patients have migraine with aura (IHS 1.2). See Headache Classification Committee of the International Society. Classification and diagnostic criteria for headache disorders, cranial neuralgia and facial pain. Cephalalgia (1988); 8: (Supp. 7): 1-96.

The IHS has defined prodromal symptoms as non-aura symptoms signaling the onset of a migraine attack. The symptoms typically occur a few hours to 48 hours before the onset of the headache phase of the migraine. Headache phase of migraine as used herein means the point in time when head pain is perceived by the sufferer. Prodrome or premonitory symptoms may occur in migraine with (IHS 1.1) and migraine without aura (IHS 1.2). The IHS prefers the term premonitory symptoms over prodrome due to historical use of prodrome to describe aura. Prodrome symptoms as used herein is synonymous to premonitory symptoms. See Headache Classification. Committee of the International Society. Classification and diagnostic criteria for headache disorders, cranial neuralgia and facial pain. Cephalalgia (1988); 8: (Supp. 7): 1-96.

Prodrome or premonitory symptoms may have physical and mental components. The symptoms have been classified by clinical presentation as excitatory and inhibitory symptoms. Excitatory symptoms include, but are not limited to, irritability, euphoria (being ‘high’), physical hyperactivity, excessive yawning, excessive sleepiness, increased sensitivity to light and sound, and craving for foods. Inhibitory symptoms include, but are not limited to, depression, mental withdrawal, behaviour sluggishness, feeling tired, poor concentration, muscle weakness, anorexia and fluid retention. See Headache Classification Committee of the International Society. Classification and diagnostic criteria for headache disorders, cranial neuralgia and facial pain. Cephalalgia (1988); 8: (Supp. 7): 1-96 and Anthony M, Rasmussen B K. In: Olesen J, Tfelt-Hansen P, Welch K M A (eds). The Headaches. New York: Raven Press, Ltd, 1993: 256-257. Prodrome/premonitory symptoms have been estimated to occur in up to 88% of migraine patients. See supra, Rasmussen.

Thus, it is desirable to provide an anti-migraine agent useful for the prevention of the headache phase of the migraine. It is further desirable to be able to predict the onset of migraine before the head pain actually occurs and thereby permit the prophylactic administration of medicine.

The Automated Neuropsychological Assessment Metrics (ANAM) is a set of standardized batteries of cognitive tests, modified by neuropsycnologists in the U.S. Armed Forces for precise measurement of cognitive processing efficiency of military personnel. The tests assess sustained concentration and attention, mental flexibility, spatial processing, cognitive processing efficiency, mood, arousal/fatigue level, and short-term, long-term and working memory. The ANAM is now in the public domain. The most recent version is ANAM V3.IIa/96 which includes the following battery of tests:

1. Subject Demographics Form

2. Stanford Sleepiness or Sleep/Fatigue Scale

3. Mood Scale 2

4. Simple and Two-Choice Reaction Time

5. Sternberg Memory Search Tasks

6. Running Memory Continuous Performance Task

7. Mathematical Processing Task

8. Digit Set Comparison Task

9. Logical Reasoning-Symbolic

10. Tower of Hanoi (Tower Puzzle)

11. Stroop Color/Word Interference

12. Code Substitution (Letter/Symbol Comparison)

13. Code Substitution (Immediate and Delayed Recall)

14. Spatial Processing Task (Simultaneous)

15. Matching to Sample

16. Tapping (Left and Right Index Finger)

17. Modified Orientation and Amnesia Test

It would be desirable to be able to use a subset of these time-consuming tests to predict the onset of migraine before the head pain actually occurs and thereby permit the prophylactic administration of medicine.

The present invention is directed to meeting one or more of the above-stated desirable objectives.

### SUMMARY OF THE INVENTION

The present invention further provides a preemptive prophylaxis migraine method using the following cognitive tests: Simple Reaction Time; Running Memory Continuous Performance Task; Matching to Sample; Mathematical Processing Task; and interpreting the results as a percent of baseline indicator of need for prophylaxis. Preferably the tests are administered in the listed sequence. Advantageously the tests are preceded by the Stanford Sleepiness Scale and Mood Scale 2 tests.

In a preferred arrangement there is provided a preemptive prophylaxis migraine device including a microprocessor having a memory, a battery of tests loaded into the memory of the microprocessor and including a Simple Reaction Time, a Running Memory Continuous Performance Task, a Matching to Sample, and a Mathematical Processing Task; means for computing the score on a trial of these tests to establish a baseline and for storing the baseline in the memory; the means for computing being operative for computing the score of a subsequent trial of the tests and comparing the same to the stored baseline; and means for indicating a cognitive change.

In one aspect of the invention, a method is provided for preventing the headache phase of migraine in a human comprising administration of an anti-convulsant to said human exhibiting prodrome symptoms of migraine.

These and other objects, aspects, features and advantages of the present invention will become apparent from the following detailed description when taken in conjunction with the referenced drawings.

### BRIEF DESCRIPTION OF THE DRAWINGS

Reference is now made more particularly to the drawings which illustrate the best presently known mode of carrying out the invention and wherein similar reference characters indicate the same parts throughout the views.

The accompanying drawings illustrate two devices and one method for carrying out the present invention and wherein:

FIG. 1 is a plan view of a hand-held computer which is one apparatus for determining cognitive change in a human;

FIG. 1A is a plan view of a palm-top type computer which is another apparatus for determining cognitive change in a human;

FIG. 2 is a flow chart illustrating the steps and sequence of a method for performing preemptive prophylaxis of migraine; and FIG. 3 is a chart illustrating the therapeutic phases of migraine and including the treatment options most suitable to each phase of Migraine.

### DETAILED DESCRIPTION

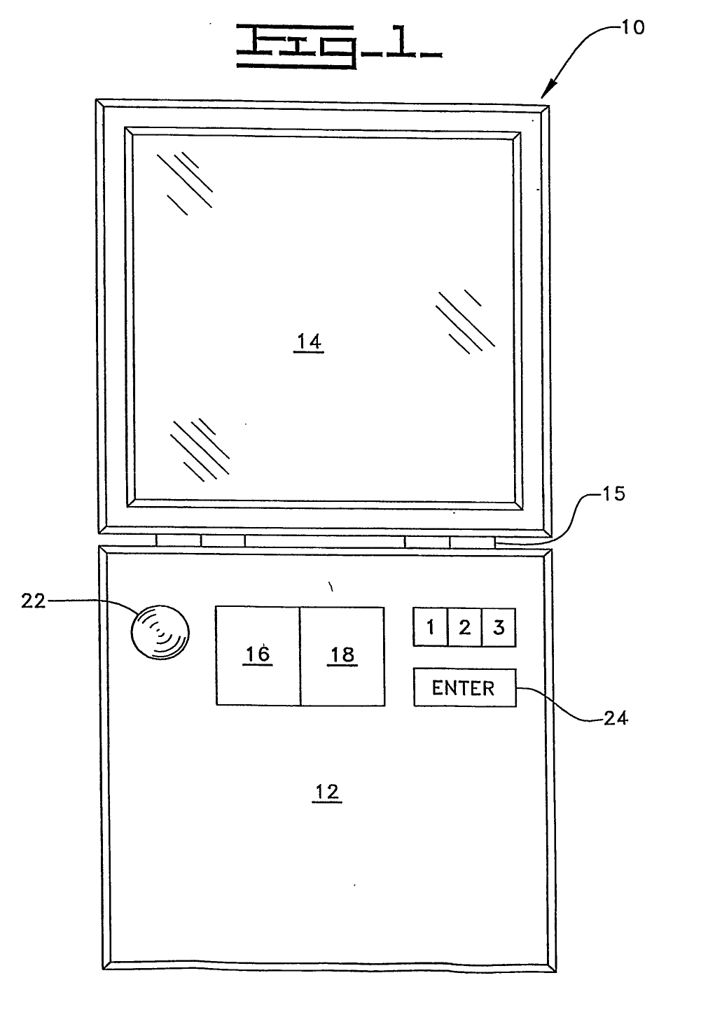

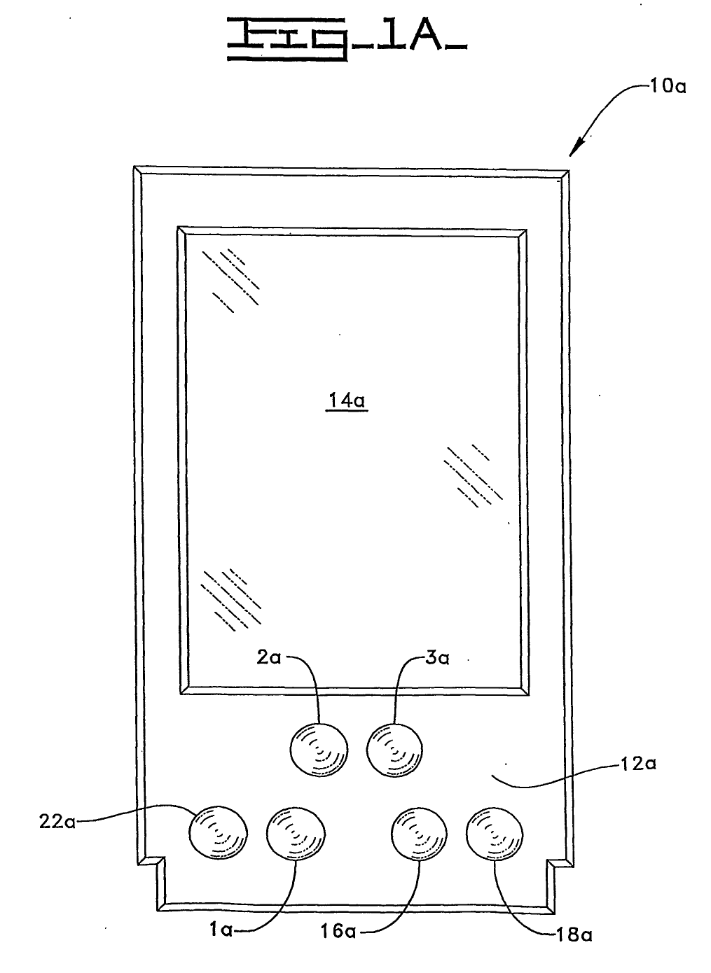

FIG. 1 shows a preemptive prophylaxis of migraine device in the form of a hand-held computer, generally designated 10, and having a key pad 12 and a screen 14 which advantageously is at least four inches (10.16 cm.) square. A hinge 15 is provided so the screen 14 may be conveniently folded down upon the key pad 12 for storage or transporting. When open the computer 10 is conveniently about 5″×9″ (12.7 cm. by 22.86 cm.) in size. The key pad 12 has a built-in set of two mouse buttons 16,18, a start/stop or on/off button 22, an enter key 24, and Mood Scale 2 keys 1, 2 and 3. As used herein the terms “buttons” and “keys” are intended to mean the same thing. The computer 10 contains memory chips (not shown) which have a set of programmed cognitive tests 103-106 (hereafter described) and which record a person's performance time in milliseconds on those tests. The computer program uses the score in milliseconds on the third trial of these cognitive tests as a baseline measurement, which is converted to a stanine score. Subsequent trials are similarly scored and converted to stanine.

FIG. 1A shows a palm-top type computer 10a which, when programmed with the cognitive tests 103-106, performs the same functions as hand-held computer 10. Accordingly, the same functional parts identified in FIG. 1, are identified in FIG. 1A with the same numerals and the letter “a”. Further description is deemed unnecessary. It is believed that the largest palm-top computer now available is 7.8 inches (19.81 cm.) long and the screen 14a is not as large as the desired four inches (10.16 cm.) square. However, this deficiency is offset by the savings in using mass produced devices.

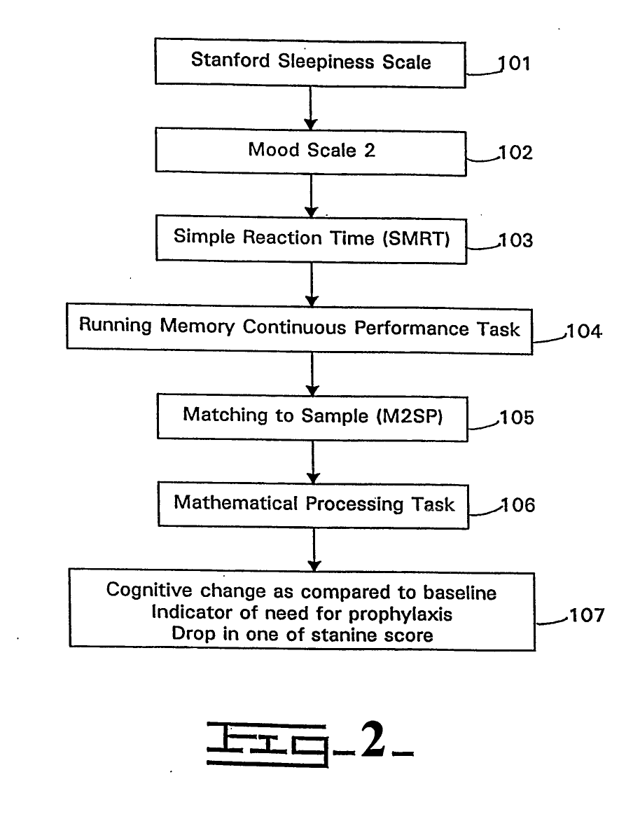

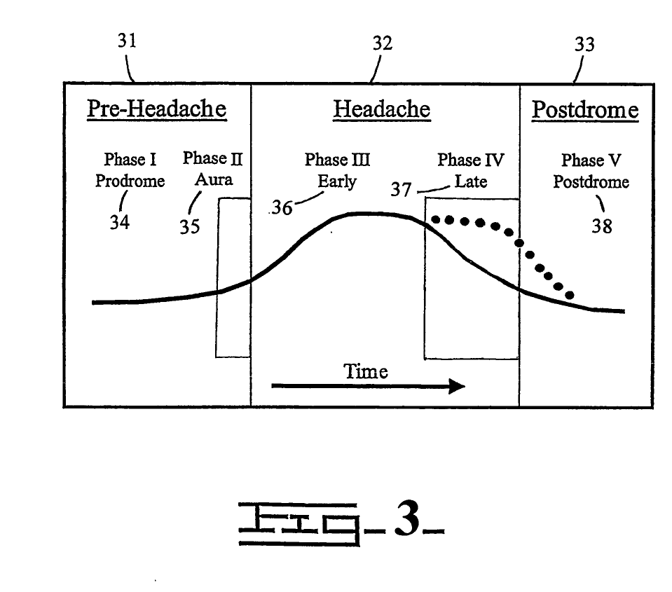

FIG. 2 shows the sequence of the method for measuring cognitive processing efficiency. From the seventeen tests of the original ANAM, four subtests were selected and sequenced for measuring cognitive processing efficiency of migraine sufferers, as follows:

1. Simple Reaction Time (SMRT), 103

2. Running Memory Continuous Performance Task (CPT), 104

3. Matching to Sample (M2SP), 105

4. Mathematical Processing Task (MATH), 106.

Also included are two preliminary measures of alertness and mood that are also part of the ANAM:

1. Stanford Sleepiness Scale, 101

2. Mood Scale 2,102.

Description of Subtests:

1. The first step 101 is Stanford Sleepiness Scale which consists of seven statements that describe the present state of alertness or sleepiness and are numbered from one to seven, with one being highly alert and seven being close to sleep. Individuals rate their level of alertness prior to taking the first subtest of the battery. It provides a way to monitor fatigue over the course of repeated measures. Subjective ratings may be correlated with measured performance.

2. The second step 102 is Mood Scale 2 which consists of a list of thirty-six adjectives that are rated on a three-point scale. Using mouse button 16 participants respond to each adjective by indicating “yes,” “moderately,” or “no,” based on how they feel at the present time. The Mood Scale 2 categories include anger, happiness, fear (anxiety), depression, activity, and fatigue.

3. The third step 103 is Simple Reaction Time (SMRT) which presents a simple stimulus on the screen (*). In response, the individual presses the mouse button 16 each time the stimulus appears. The Reaction Time measures the speed of the motor response, the peripheral nerve conduction velocity. This represents the “hardware” of the nervous system in terms of input, followed by motor response. Actual cognitive processing time is not involved in this test.

4. The fourth step 104 is Running Memory Continuous Performance Test (CPT) which is a continuous letter comparison task. A randomized sequence of upper-case letters, A through Z, is presented one at a time in the center of the computer screen 14. The person presses button 16 if the letter on the screen matches the letter that immediately preceded it; and different button 18 if the letter on the screen is different than the immediately preceding letter. The task lasts approximately five minutes. The CPT was specifically designed to assess components of memory, attention, efficiency and consistency. This task is forced paced, with individuals having only a brief time in which to respond.

5. The fifth step 105 is Matching to Sample (M2SP) and consists of a number of trials that begins with a first design being presented in the center of the screen 14 for three seconds, followed by a showing that contains two designs. The person matches one of the two designs with the first design or sample by pressing the appropriate button 16 or 18. The design is a 4×4 checkerboard and varies by the number of cells that are shaded from one cell through twelve cells.

6. The sixth step 106 is Mathematical Processing (MATH) and involves arithmetic problems presented in the middle of the screen 14. Working from left to right, the person solves the addition and subtraction and decides if the answer is greater or less than the number 5.

As indicated, the scores are recorded by the computer 10 and the score on the third trial of these sequenced cognitive tests 103-106 are used as the baseline measurement. Subsequent trials measure cognitive change as compared to baseline. A drop of one in stanine score is an indicator of the onset of migraine and an indicator of need for prophylaxis. See FIG. 2, 107. This was empirically determined by the following research. The preemptive prophylaxis of migraine method was used to measure cognitive deficiency during a migraine in each of a group often migraineurs. The method was used to measure the return of cognitive efficiency after injection of sumatriptan, an anti-migraine medication, in each of the group often migraineurs. The method measured cognitive change, compared to the baseline stanine score, that predicted the onset of a migraine.

The above described preemptive prophylaxis of migraine device and method allows a migraine sufferer to take medication to preempt the occurrence of head pain, associated symptoms and accompanying disability.

It will be appreciated that the precise dose of medication administered to prevent the headache phase of migraine may depend on the particular compound used, the age and condition of the patient and the frequency and route of administration and will be at the ultimate discretion of the attendant physician.

FIG. 3 shows the phases of migraine. The entire migraine event is roughly divided into three time periods: pre-headache 31, headache 32 and postdrome 33. These time periods are further divided into phases. The pre-headache period is composed of Phase I, prodrome 34, and Phase II, aura 35. The headache period is divided into Phase III, early headache 36, and Phase IV, late headache 37. Finally, Phase V, postdrome 38, encompasses the entire postdrome period.

During the prodrome phase 4 of migraine, identified in FIG. 3, anti-convulsant medications are utilized to preclude the onset of the headache phase. Examples of suitable anti-convulsants, with their brand name equivalents, include tiagabine hydrochloride (Gabitril®), gabapentin (Neuronting), phenytoin sodium (Dilantin®), carbamazepine (Carbatrol®), divalproex sodium (Depakote®), felbamate (Felbatol®), levetiracetam (Keppra®, primidone (Mysoline®), carbamazepine (Tegretol®), topiramate (Topamax®), oxcarbazepine (Trileptal®), zonisamide (Zonegran®), lamotrigine (Lamictal®), methsuximide (Celontin®), and thosuximide (Zarontin®). It is contemplated that any of these anti-convulsant medications, and combinations thereof may be used as a prophylaxis to preclude the onset of migraine.

Basically, the preemptive prophylaxis method contemplated herein includes determining the impending onset of migraine and administering an anti-convulsant medication to preclude the bnset of migraine. As used herein, the term “preclude” is intended to include reducing adverse effects of or reducing the duration of migraine. Advantageously, the onset is determined during the prodrome phase and the determination is made via tests to determine cognitive deficiency. Advantageously, the medication is one or more of known anti-convulsant medications.

Other objects, features and advantages of the present invention will be apparent to those skilled in the art. While preferred medications and steps of the method have been illustrated and described, this had been by way of illustration and the invention should not be limited except as required by the scope of the appended claims.

What is claimed is:

1. The use of an anti-convulsant in the manufacture of a medicament for administration to a human during a prodromal period of migraine in order to prevent the headache phase of migraine.

2. The use according to claim 1 wherein the anti-convulsant is selected from the group consisting of tiagabine hydrocholoride, gabapentin, phenytoin sodium, carbamazepine, divalproex sodium, felbamate, levetiracetam, primidone, topiramate, oxcarbazepine, zonisamide, lamotrigine, methsuximide, and thosuximide.

3. (canceled)

4. (canceled)

5. A preemptive prophylaxis migraine method, comprising the steps of:
determining prodromal symptoms of migraine in a human and thereby identifying a prodromal period of migraine; and
administering a migraine headache phase-preventing amount of an anti-convulsant to the human during the prodromal period of migraine.

6. A preemptive prophylaxis migraine method as set forth in claim 5, wherein the step of administering an anti-convulsant further comprises selecting the anti-convulsant from the group consisting of tiagabine hydrocholoride, gabapentin, phenytoin sodium, carbamazepine, divalproex sodium, felbamate, levetiracetam, primidone, topiramate, oxcarbazepine, zonisamide, lamotrigine, methsuximide, and thosuximide.

7. (canceled)

8. (canceled)

9. A preemptive prophylaxis migraine method as set forth in claim 5, wherein the step of determining prodromal symptoms further comprises performing cognitive tests to measure cognitive changes in the human to determine prodromal symptoms of migraine and thereby identifying the prodromal period of migraine.

10. A preemptive prophylaxis migraine method as set forth in claim 9, wherein the step of performing cognitive tests further comprises the steps of:
establishing a baseline indicator from the performed cognitive tests;
repeating the tests; and
interpreting the results of the repeated tests as a percent of the baseline indicator of need for prophylaxis.

11. A preemptive prophylaxis migraine method as set forth in claim 10, wherein the step of administering an anti-convulsant further comprises selecting the anti-convulsant from the group consisting of tiagabine hydrocholoride, gabapentin, phenytoin sodium, carbamazepine, divalproex sodium, felbamate, levetiracetam, primidone, topiramate, oxcarbazepine, zonisamide, lamotrigine, methsuximide, and thosuximide.

12. A preemptive prophylaxis migraine method as set forth in claim 11, wherein the step of performing cognitive tests further comprises performing tests selected from the group consisting of: a Simple Reaction Time test, a Running Memory Continuous Performance Task, a Matching to Sample test, and a Mathematical Processing Task.
